(19) United States
(12) Patent Application Publication
(10) Pub. No.: US 2004/0133060 A1
(43) Pub. Date: Jul. 8, 2004

(54) Application device for magnetic field treatment

(76) Inventors: Inka Wilhelm, Alling (DE); Klaus Wassweiler, Alling (DE); Bruno Koch, Weilrod (DE)

Correspondence Address: HENRY M FEIEREISEN, LLC, 350 FIFTH AVENUE, SUITE 4714, NEW YORK, NY, 10118, US

(21) Appl. No.: 10/701,395

(22) Filed: Nov. 4, 2003

Related U.S. Application Data
(60) Provisional application No. 60/430,205, filed on Dec. 2, 2002.

(30) Foreign Application Priority Data
Nov. 4, 2002 (DE) 202 16 970.7

(51) Int. Cl. A61N 2/04

(52) U.S. Cl. 600/14; 600/9

(57) ABSTRACT

The present invention relates to a magnetic field treatment. In particular, the present invention relates to an application device for providing a magnetic field in an inner space of a housing. At least one surface of the housing is equipped with equipment for generating the magnetic field. Thus, animals or humans located in the inner space of the housing are exposed to the magnetic field, which may have a curative effect on the humans or animals.

### CROSS-REFERENCES TO RELATED APPLICATIONS

This application claims the benefit of prior filed U.S. provisional Application No. 60/430,205, filed Dec. 2, 2002, pursuant to 35 U.S.C. 119(e), the disclosure of which is incorporated herein by reference.

This application claims the priority of German Patent Application, Serial No. 202 16 970.7, filed Nov. 4, 2002, pursuant to 35 U.S.C. 119(a)-(d), the disclosure of which is incorporated herein by reference.

### TECHNICAL FIELD

This invention concerns a device for generating a magnetic field in an interior space of a housing.

### BACKGROUND TO THE INVENTION

The earth on which we live represents a huge magnet. Although the magnetic field produced by the earth is relatively weak—in our latitudes it shows a magnetic flow density of about 0.5 gauss—nevertheless, it influences a large number of biological processes. For example, many animals, particularly migratory birds, possess a sense organ which thanks to this magnetism, helps them in their spatial orientation. Man does not, however, subjectively feel the earth's magnetism, is, nevertheless influenced imperceptibly and unconsciously by the magnetic field of the earth.

The magnetism phenomenon has been known since ancient times. Even in old China the knowledge of the magnetic field was used for curative purposes for which permanent iron magnets were used. Also from ancient Egypt, Greece and Rome there is evidence that already at that time they fell back on the healing effect of magnetic fields for the treatment of diseases and symptoms. In occidental cultural circles first evidence for the treatment of diseases using magnetic fields can be found from about 1500 AD. Particularly the famous doctor Paracelsus made his name through this. At the end of the 19^{th} century artificial magnetic fields were produced for the first time. For this purpose electric currents were run through iron rings or coils made of several wire windings. Modern magnetic field treatment is also today still based on this principle. Most of the devices for magnetic field treatment available today work with electrically produced pulsating magnetic fields which are produced through coils with wire wound round them.

The mode of action of the magnetic field treatment can be explained as follows: the magnetic or electromagnetic waves flow through the human or animal tissue and in this way act on the body cells. At the same time positively and negatively charged particles which can be found everywhere in the human or animal bodies are affected positively and are newly aligned. The cell formation is thereby improved, the metabolism is stimulated and the circulation boosted. Pain receptors which are responsible for pain perception are inhibited which means that the pain is relieved.

As it has been known since the sixties that bones are piezoelectric, which means that with every load change from the bones currents are produced, today it seems certain that the self-produced currents are fundamentally involved in the construction and the preservation of healthy bones. This piezo effect of the bones has since been made use of for therapeutic purposes by artificially producing normal mechanical load changes in the bones, as, for example, in sport and in motion gymnastics without putting weight on the joints. The complete electromagnetic energy is transferred through variable frequencies and impulses, through which, for example, bone fractures heal more quickly.

Moreover magnetic fields raise temperature (thermomagnetic effect), in the cells the polarisation is changed by which the ions are arranged in a certain way and are newly aligned and through which the cell membrane potential is positively influenced and improved. In addition through the effect of the magnetic fields the photonemission of the DNA can be influenced and with this the intracellular communication as well as the cell metabolism.

The number of positive influences and effects on the human and animal organism seems to be almost unlimited and extends from orthopaedic ranges of application like, for example, arthritis, arthrosis or discogenic diseases through internal areas of application like, for example, bronchitis, multiple sclerosis or muscular atrophy to dermatological areas of application like, for example, acne or allergies to gynaecological areas of application such as dysmenorrhea or salpingitis to name but a few. This wealth of possible areas of application leads, especially in the current age of the wellness and fitness boom, to a real rush on possible applications for the magnetic field treatment. In the modern practice of application, for example, a large number of devices are known for local treatment with the help of magnetic field therapy. Such devices are, for example, magnetic field blankets, cushions, hand apparatus, sticks, rings as well as magnetic field bandages and much more. All these known devices and pieces of equipment are, however, impaired by problems regarding their handling. Frequently this equipment is either unwieldy and poor to handle or they force the user to be treated on a hospital ward or at the very least as an out-patient in order to devote himself exclusively to the magnetic field treatment and release himself for the duration of the application of all remaining jobs and activities. The patient to be treated has to, therefore, on application of most of the devices associated with the current level of technology, frequently take extra time in order to enjoy the curative effect of the magnetic fields.

The application of magnetic fields in animals for their treatment with those devices associated with the current level of technology also turns out to be immensely difficult, as these, in order to be able to be treated have, in many cases, to be anaesthetised, tranquillised or held for the treatment time which can turn out to be extremely difficult in individual cases. In particular animals can react very anxiously or aggressively when they are brought into, for example, in-patient magnetic field treatment equipment which often makes their treatment impossible.

### DESCRIPTION OF INVENTION

Therefore, underlying this invention was the problem to make available an application device for veterinary and human medical usage with which the problems known from the current level of technology could be overcome, i.e. to make available an application device which facilitates more easily the enjoyment of the curative effect of the magnetic fields. This technical problem is solved in accordance with a first aspect of this invention with an application device for the treatment of diseases in humans or animals with an inner cavity surrounded by a casing. In this connection the casing shows at least a side, wall, roof or surface which is equipped with settings to produce a magnetic field in the inner cavity of the casing. With the magnetic field produced in this way in the inner cavity of the casing of the application device humans or animals located in it can easily be treated. An application device such as this proves to be particularly advantageous in as much as, with this, those problems associated with the current level of technology can be overcome. In particular in the treatment of animals this device according to the invention proves to be particularly advantageous as these can be more easily treated with this device according to the invention. In those application devices associated with the current level of technology like, for example, hand apparatus, rings, bandages, cushions or pools the problem was frequently that those animals to be treated could not easily be tranquillised in order to be subject to the magnetic field treatment.

Through these devices according to the invention this problem has now been solved as the coils which are the necessary equipment for the production of a magnetic field with, for example, electric currents flowing through them are arranged in at least a few of the inner wall surfaces of the respective casing. In this way those animals to be treated can undergo the magnetic treatment directly in their stable or cage without having to be specially held or anaesthetised. A particularly appropriate form of design consists of replacing, for example, the magnetic field coils with permanent magnets. Such a application device with permanent magnets proves to be particularly convenient as such a device can, for example, be implemented also in the wild where the energy necessary for the production of electrically produced magnetic fields is not easily available.

This invention proves, however, to be particularly favourable as well in the human medical area of application as, on the one hand, through this device in-patient or at least out-patient treatments in an appropriate therapeutic practice become, to the greatest extent possible, superfluous in as much as the casing for the application device can be designed as, for example, a home sauna or another type of cubicle such as, for example, a sort of solarium or a magnetic field cubicle. In the side or top and/or underneath the inner wall surfaces of this the necessary equipment is provided for the production of a magnetic field. One particular advantage is the fact that sometimes the really large amount of time which is normally necessary for an in-patient or at least an out-patient curative treatment is minimised or at least reduced as the person to be treated does not now have to just go specially to the appropriate therapeutic practice but can carry out the treatment completely as a sideline, for example, during a solarium or sauna at home. This shows the additional positive effect that the person to be treated accordingly undergoes a treatment session more often as this is now no longer associated with very tiresome extra time but can happen at the same time as something else.

This time optimisation effect can of course be drastically intensified by furnishing the application device according to the invention in addition with equipment in order to carry out other types of treatment at the same time as the magnetic field treatment like, for example, phototherapy (in particular infrared therapy) a sound therapy or aromatherapy to name but a few. Such a multifunctional application device has, for example, in the side inner wall surfaces treatment heads, loud speakers or scent nozzles.

Instead of building the equipment necessary for the production of the magnetic field into the inner wall surfaces of a home sauna it is, of course, also possible to provide for this equipment in other places in which the person to be treated normally is stationary for a length of time. For example, equipment for the production of a magnetic field can be provided for in or on the floor and/or the ceiling of an office which a person stays in for a long time. In the same way the seat or the back-rest of an office chair can be fitted with permanent magnets and coils which electric current runs through so that a person sitting on it can at the same time be subject to the curative effect of the magnetic fields.

In accordance with a further aspect of the invention the inner wall surfaces of the casing of the application device are equipped with pairs of coils with electric current running through them. With such an arrangement a very homogenous magnetic field is produced in the inner cavity of the application device which brings a particularly curative effect with it. In the same way it is, however, quite conceivable to furnish, for example, the floor surface of the cage or, for example, of a dog's kennel with the necessary equipment for the production of a magnetic field. This can in particular be especially convenient in veterinary medical usage cases as most of the animals lie flat on the floor in order to have a rest or go to sleep. This device according to the invention, therefore, is quite literally appropriate for “healthy sleeping”.

As with the design form mentioned before with the equipment for the production of a magnetic field arranged in the floor of the animal's cage it is not difficult to recognise that it is, of course, also advantageous to provide for the appropriate device for the production of magnetic fields in a bed stead or a mattress as through this the curative effect of the magnetic field can also be applied to the sleeping human.

As already mentioned before it is particularly desirable to produce as homogenous magnetic fields as possible for treatment purposes. In accordance with a further design form of the invention such a homogenous magnetic field can be produced with a coil with electric current running through it whose windings stretch directly around the inner wall surface of the casing of the application device whilst the individual windings on the coil are basically parallel to one another.

In accordance with a further aspect of the invention at least one inner wall surface of the casing of the application device is fitted with one or more separate coils locally in order to produce a particularly pronounced magnetic field in this area. If these coils are in addition, furthermore, installed movably on to the inner wall surfaces this brings the further advantage that, for example, independent of the size or the condition of the animal or person to be treated the coils can be individually arranged in order to locally run through the area to be treated with a local magnetic field.

In accordance with a further aspect of the invention this proves to be especially advantageous in particular through the respective flow density of the magnetic field which can be made available with the device. Especially compared to the current level of technology a fundamental improvement can be seen here as those devices known like, for example, magnetic field blankets or bandages can merely provide a maximum flow density of approx. 0.5-0.8 mT (or 6-8 Gauss). The flow density produced with the aid of the known devices, therefore, only exceeds the natural magnetic field of the earth slightly so the effect of these devices can, at least, be seriously questioned. With the help of the application device according to the invention, on the other hand, it is, however, possible to produce magnetic fields in the inner cavity of the device with a flow density of up to 1000 mT (or 10000 Gauss) which makes itself felt in particular through a quicker recovery success. What has proved to be very appropriate above all is to set the flow density of the magnetic field in the inner cavity of the device at a strength of about 500 to 600 mT (5000 to 6000 Gauss). In order to achieve particularly excellent recovery successes a particularly favoured field intensity of the magnetic field is an intensity of about 100 mT (1000 Gauss).

Over and above that the flow density in the whole area is smoothly adjustable up to 1000 mT in order to individually fit the needs of each patient. Moreover, it is possible to pulse the magnetic field produced by the current running through the coils where the pulse frequency is smoothly adjustable between 1 and 200 Hz. Through this the piezo effect of the bones can be utilised for therapeutic purposes by artificially producing the normal mechanical load change in the bones as for example, in sport and motion gymnastics without, however, putting damaging weight on the joints. This effect is further strengthened on reversing the field direction of the respective magnetic fields by reversing the polarity.

A further user friendly option of this device according to the invention is that the magnetic field supplied by the application device can be selected in terms of it's intensity, frequency as well as further characterising features of the magnetic field from appropriate indication specific programmes. The user does not have to, therefore, individually manually and with a large amount of effort set the characterising features for the respective case of application but instead it is sufficient to select a certain programme which is suited to the respective indication.

It can prove to be particularly advantageous especially in veterinary medical cases of application to design the application device as a mobile unit. In this way it can be very favourable, for example, for the transportation of horses to fit a horse box, whose inner wall surfaces according to the invention are provided with devices for the production of a magnetic field, with wheels in order to make the horse box itself easily transportable. In the same way it is, however, conceivable, for example, to fit a conventional horse box according to the invention with coils with current running through them or permanent magnets. In order to make use of such a transportable device fitted with coils without the necessary current for the production of a magnetic field being available the application device is fitted with a rechargeable battery which provides the necessary energy.

The casing itself can be manufactured from any non magnetic material. It has proven to be particularly appropriate, however, to make the casing out of wood where the individual wooden components are glued together at the connections. Due to the wooden construction the inner cavity of the casing becomes particularly snug both for the animal as well as for humans—here regarding a sauna—which has an effect on the well-being and because of that on the time which the person concerned likes to spend in the device and with that it has a direct effect on the treatment success.

Moreover, it should be mentioned that, due to its inherent characteristics, the application device according to the invention is, in particular, very suitable for exposing animals or humans to the magnetic field produced by the application device which are in the inner cavity of the casing or at least parts of them in order to contribute to the relief of pain or cure of the respective disease. An appropriate procedure for the treatment, alleviation, cure or therapy in man or animal can be covered, for example, by the following steps:

(a) Supply of the application device according to the invention

(b) Open, if available, insertion opening leading to the inner cavity of the application device

(c) Introduction of the human or animal to be treated or at least a part of it into the inner cavity of the application device

(d) If available alignment of at least one separate piece of equipment for the production of a magnetic field with the part of the animal or human to be treated

(e) Adjust the flow density of the magnetic field of the application device and/or of the separate magnetic field which is provided through the separate equipment for the production of a magnetic field to an appropriate indication specific intensity

(f) Irradiation and/or flowing through the area to be treated

Regarding these procedure steps it should be noted that the order listed here of the individual steps is not compelling but individual procedure steps can simply be exchanged for each other or they can be shifted. In this way it is, for example, completely conceivable that step (e) (adjusting the flow density of the magnetic field) can occur before step (b), namely opening the inner cavity of the application device.

### BRIEF DESCRIPTION OF THE DRAWINGS

In the following several design examples of the invention will be more closely described with reference to the enclosed drawings for better understanding and for further clarification

FIG. 1 is a perspective view of an application device according to the invention in the form of a dog kennel;

FIG. 2 shows the application device according to FIG. 1 with further features not discernible in FIG. 1

FIG. 3 shows further features of the application device according to the invention according to FIG. 1

In all figures the same parts are marked with corresponding references.

### DETAILED DESCRIPTION OF AN EXEMPLARY EMBODIMENT

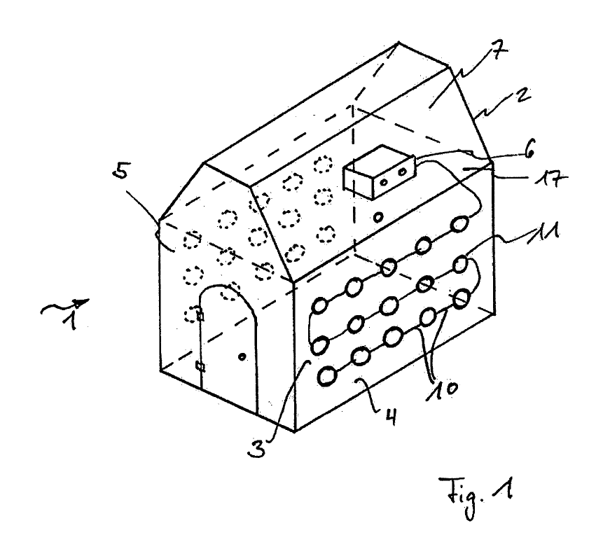

FIG. 1 shows a first exemplary embodiment of the application device 1 according to the invention in the form of a dog kennel. The kennel consists of a casing 2 with a roof space 7. The roof space 7 is separated from the inner cavity 3 of the casing 2 of the kennel by a false floor 17. The casing 2 shows two side inner wall surfaces 4, 5 which are equipped with a system 10 with coils 11 with current running through them. The coils can at the same time, of course, be arranged also in the floor or roof surfaces of the casing 2. The system 10 of coils 11 is provided, by a control unit 6, which is arranged in the roof space 7 with the necessary energy for the production of a magnetic field in the inner cavity 3 of the casing 2. A particular feature of the control unit 6 is an operating device 19 to influence the intensity of the magnetic field manually, for example, via a tuning knob. Moreover the pulse frequency as well as further characterising features of the magnetic field can be set via the control unit 6. Via the operational devices 19 an indication specific programme can also be set, the respective magnetic field intensity and frequency is accordingly controlled.

As can be easily recognised in FIG. 1 the inner cavity 3 of the dog's kennel is not effected by the technical devices for the production of the magnetic field in inner cavity 3. A dog to be treated is, therefore, not disturbed by the technical equipment in the dog's kennel and therefore, will not repel the application device as is frequently seen in those devices associated with the current level of technology. A dog, once in the inner cavity 3 of the application device 1, will not therefore, feel as if it is in a strange or unfamiliar treatment atmosphere and therefore, allow itself to be treated easily with the curative effect of the magnetic field which is produced in the inner cavity 3 of casing 2 through system 10 with coils 11, without further assistance being necessary, for example, tranquillising or holding. Instead of a dog's kennel with the casing it can of course be any other form of animal enclosure or stable like, for example, a rabbit hutch or a horse box. Regarding the human medical application area the casing could, for example, be a sauna or a small room. It is also, for example, conceivable to equip the side walls and/or the floor of a swimming pool with coils with current running through them which, for example, proves to be particularly advantageous for people who are for treatment purposes anyway, for example, for hydrogymnastics in a swimming pool. In this way these patients can quite literally kill two birds with one stone by, for example, on the one hand doing hydrogymnastics for treating their back complaints and on the other hand at the same time exposing themselves to the curative effect of the magnetic fields. The casing can also, for example, be shaped like a sun bed or solarium. A simple cuboid body can be designed as casing as well. Even a cubicle is conceivable as a casing.

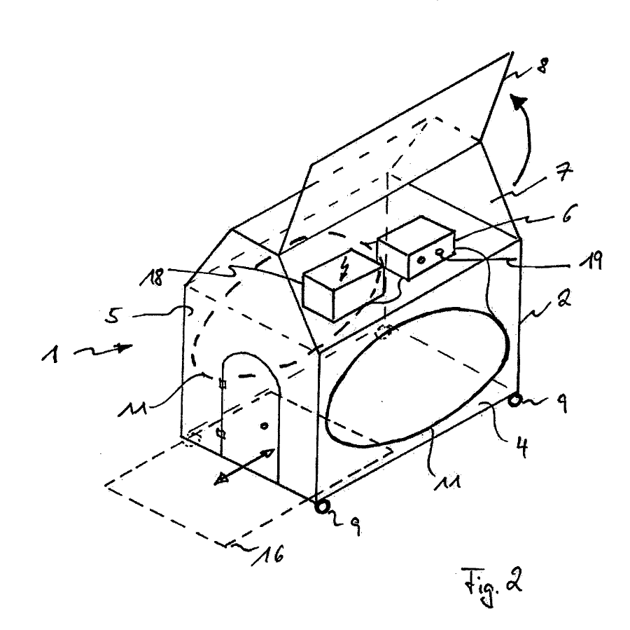

FIG. 2 shows some additional features of an application device according to the invention as already shown in FIG. 1. FIG. 2 shows as well as FIG. 1 also an application device in the form of a dog's kennel consisting of a casing 2 with a roof space 7. Instead of system 10 made of several coils 11 as shown in FIG. 1, here the inner wall surfaces 4,5 are equipped with one coil 11 each, whose windings stretch to the level of the respective limiting surfaces 4,5. The roof space 7 contains a control unit 6 for the manipulation of the magnetic fields containing as well a rechargeable battery 18, which provides the control unit 6 with current. In order to make the control unit 6 and the rechargeable battery 18 easily accessible from outside the application device 1 in the form of a dog's kennel 2 contains a roof top 8 which can be opened upwards through which the control units 6 can be easily accessible. Over and above that the application device is equipped on the floor areas with several rollers or wheels 9 which allow it to easily transport the device 1 or to move it. As can be recognised in FIG. 2 the kennel contains a insertion 16 in the floor which is suitable for easy removal of the straw in the inner cavity 3 of casing 2.

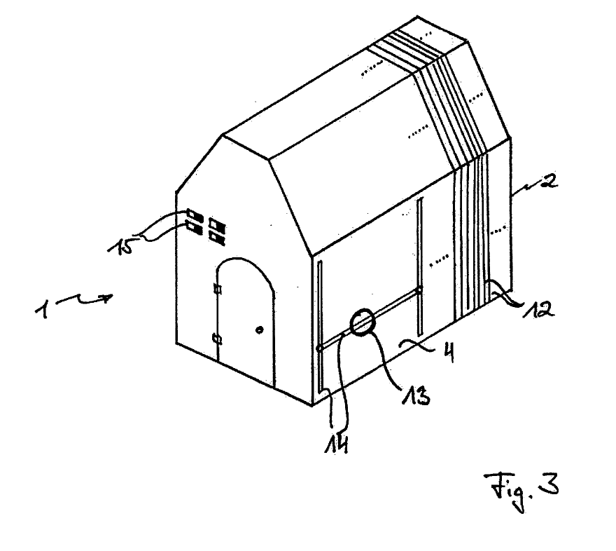

FIG. 3 shows further possibilities for the equipping of an application device according to the invention 1 with equipment for the production of magnetic fields. As can be recognised in the rear section of the application device 1 the whole casing 2 is designed with a large number of windings 12 on a coil. The windings 12 stretch around the whole circumference of the casing 2 and are fundamentally parallel to one another. This large number of windings 12 together result in a huge coil which, in this way, at least partly surrounds the whole casing 2. The coil, consisting of windings 12, is in turn connected to equipment for controlling the current which runs through the coil. Due to such a “capacity coil” in the inner cavity 3 of the application device 1 very homogenous magnetic fields can be produced which allows a particularly convenient usage. As can be recognised in FIG. 3 the casing 2 of the application device 1 and the limiting surfaces 4 are designed with rails on which a coil with electric current running through it is designed to slide. This sliding coil design is particularly suitable to fulfil the individual needs of the animal to be treated. With the help of this device it is possible, to treat quite specifically and locally certain parts of the animal to be treated. Of course it is possible also to design several coils of this kind to slide or to provide for other devices which allow the shifting of a coil 13.

Furthermore, in FIG. 3 on the front of the casing four permanent magnets 15 are arranged. Even if only 4 permanent magnets 15 are arranged here it is of course possible at any time to design the whole casing or certain parts of it with such permanent magnets to produce a magnetic field in the inner cavity 3 of the casing 2 for magnetic field therapy. These permanent magnets 15 could of course also be put on to rails 14 or other appropriate devices sliding on the casing 2.

One or several further application devices can be provided for in the casing which is particularly advantageous. For instance lighting devices like, for example, treatment heads or LEDs with various colours for usage in a light therapy can be planned, sound aids for a sound therapy, an aroma production equipment for an aromatherapy or infrared treatment heads for thermotherapy.

The equipment for generating the magnetic field may be provided in the walls, roof or base of the housing. Furthermore, the equipment may also be accommodated in the walls, the roof or the base of the housing. Also, the equipment may be provided on outer and/or inner surfaces of the roof, the walls or the base of the housing.

While the invention has been illustrated and described in connection with currently preferred embodiments shown and described in detail, it is not intended to be limited to the details shown since various modifications and structural changes may be made without departing in any way from the spirit of the present invention. The embodiments were chosen and described in order to best explain the principles of the invention and practical application to thereby enable a person skilled in the art to best utilize the invention and various embodiments with various modifications as are suited to the particular use contemplated.

What is claimed as new and desired to be protected by Letters Patent is set forth in the appended claims and includes equivalents of the elements recited therein:

What is claimed is:

1. Application device for providing a magnetic field in an internal space surrounded by a housing for providing a magnetic treatment to humans or animals, wherein at least one region of the housing is fitted with equipment for the generation of the magnetic field in the internal space of the housing in order to expose the human or animal in the internal space to the magnetic field.

2. Application device according to claim 1, wherein the equipment is at least one electric coil with current running through it.

3. Application device according to claim 2, wherein the at least one coil with electric current running through it is arranged in at least one surface selected from the group of surfaces consisting of roof surfaces, floor surfaces and side surfaces.

4. Application device according to claim 2, wherein the magnetic field is generated by at least two coils with electric current running through which are arranged in or on two regions of the housing opposite each other.

5. Application device according to claim 3, wherein the magnetic field is essentially homogenous.

6. Application device according to claim 1, wherein the magnetic field is generated by several coils with electric current running through them arranged in or on a part of the housing.

7. Application device according to claim 1, wherein the magnetic field is generated by a coil whose windings extend around an outer surface of the housing.

8. Application device according to claim 1, wherein in or on at least one of the part of the housing, individual coils are arranged for the generation of a local magnetic field.

9. Application device according to claim 8, wherein the coils are fastened individually and movably.

10. Application device according to claim 2, wherein the coil is arranged with its main axles essentially vertical to the respective surface of the housing.

11. Application device according to claim 1, wherein the magnetic field has a maximum flow density of about 1000 mT.

12. Application device according to claim 1, wherein a flow density of the magnetic field is about 100 mT.

13. Application device according to claim 1, wherein a flow density of the magnetic field is in the region of between 500 mT and 600 mT.

14. Application device according to claim 1, wherein the flow density is smoothly adjustable in the region of up to about 1000 mT.

15. Application device according to claim 1, wherein the magnetic field is pulsed.

16. Application device according to claim 15, wherein a pulse frequency of the magnetic field is adjustable in the region between 1 and 200 Hz.

17. Application device according to claim 1, wherein the time intensity of the magnetic field follows a rectangle, saw tooth or sine course.

18. Application device according to claim 1, wherein a direction of streamlines of the magnetic field from one to the next impulse reverses.

19. Application device according to claim 1, wherein the flow density and the frequency of the magnetic field can be manually adjusted via a control unit which is in or on the housing.

20. Application device according to claim 19, wherein the flow density and the frequency of the magnetic field is controlled by an indication specific start-up programme which is set via the control unit.

21. Application device according to claim 1, wherein the equipment is supplied with current from an energy storage.

22. Application device according to claim 1, wherein the magnetic field is produced by at least two permanent magnets.

23. Application device according to claim 1, wherein the housing is a sauna, a magnetic field chamber or a solarium.

24. Application device according to claim 1, wherein the housing is an animal's cage or stable.

25. Application device according to claim 1, wherein the housing contains an at least partly removable side.

26. Application device according to claim 1, wherein the application device is designed as a mobile unit and comprises at least two wheels.

27. Application device according to claim 1, wherein the housing is accessible to animals or humans.

28. Application device according to claim 1, wherein the housing consists of a non-magnetic material and has no magnetic components.

29. Application device according to claim 1, wherein the housing at least partly consists of one of the materials from the group of materials consisting of wood, synthetic substance, plaster and stone.

30. Application device according to claim 1, wherein the application device is provided with means for the simultaneous implementation of other treatment types.

31. Application device according to claim 30, wherein the application device is equipped with treatment heads, loud speakers or scent nozzles.
